(19) United States
(12) Patent Application Publication
(10) Pub. No.: US 2022/0273588 A1
(43) Pub. Date: Sep. 1, 2022

(54) MONOAMINE OXIDASE B INHIBITORS FOR USE IN THE PREVENTION OR TREATMENT OF PROSTATE CARCINOMA

(71) Applicant: TOXIE EUROPE INTELLIGENS KÉMIAI SZENZOROKAT KUTATÓ, FEJLESZTÖ KORLÁTOLT FELELÖSSÉGÜ TÁRSASÁG, Budapest (HU)

(72) Inventors: Viktória GASZNERNÉ KORMOS, Keszü (HU); Tamás KÁLAI, Pécs (HU); László MANGEL, Pécs (HU); Péter MÁTYUS, Budapest (HU); Anita STEIB, Kárász (HU); Zsuzsanna TAMASIKNÉ HELYES, Pécs (HU)

(21) Appl. No.: 17/632,571

(22) PCT Filed: Aug. 5, 2020
(86) PCT No.: PCT/HU2020/050035

§ 371 Date: Feb. 3, 2022

(30) Foreign Application Priority Data
Aug. 6, 2019 (HU) P1900281

(51) Int. Cl. A61K 31/137; A61K 31/337; A61K 45/06; A61P 35/00

(52) U.S. Cl. CPC A61K 31/137; A61K 31/337; A61K 45/06; A61P 35/00

(57) ABSTRACT

The present invention provides a monoamine oxidase B (MAO-B) inhibitor compound for use in the prevention or treatment of prostate carcinoma (PCa), wherein no selective MAO-A inhibitor compound is co-administered. In the use, other agents for the treatment of PCa is administered and/or radiotherapy is used for the treatment of PCa together with or alternately with the selective compound MAO-B. Another aspect of the invention is the use of a MAO-B inhibitor compound in the manufacture of a medicament for the treatment of PCa.

The present invention relates to the use of monoamine oxidase-B (MAO-B) enzyme inhibitors, in particular selegiline and rasagiline, in the treatment of prostate carcinoma (PCa) and in the manufacture of a medicament for the treatment of PCa.

### DESCRIPTION OF THE STATE OF THE ART

PCa is one of the most common tumour disease among males, the second most common tumorous disease after lung cancer in terms of the number of new cases diagnosed nowadays. For males, prostate carcinoma is among the most common causes of death worldwide and the second most common cause of cancer death in Western society (Shih 2018); in addition, the incidence of PCa is increasing.

PCa is also an important area from a veterinary point of view, in case of dogs and horses this disease is especially notable: it can be seen that there is an increase in the incidence of prostate carcinoma among oncological diseases (https://wearethecure.org/learn-more-about-canince-cancer/canine-cancer-library/prostate-cancer/ downloaded: Aug. 1, 2019; https://ihearthorses.com/the-5-most-common-types-of-cancer-in-horses/downloaded: Aug. 1, 2019).

For the treatment of PCa, surgical, radiotherapy procedures and drug therapy (mainly chemotherapy and hormone therapy) may be considered, which are used according to international treatment guidelines depending on the type of tumor and the severity and progress of the disease (localized vs. metastatic PCa) (N. Mottet et al., Eur Urol., 2017, 71, 618-629; P. Cornford et al., Eur Urol., 2017, 71, 630-642; European Association of Urology 2018; National Comprehensive Cancer Network, 2019).

In recent years, in particular, the approach and effectiveness of the treatment of localized PCa has developed a lot. On the one hand, close monitoring without intervention seems to be appropriate for low-risk disease, and on the other hand, it is a significant achievement that radiotherapy and surgical interventions are becoming more effective through technical progress at the higher risk stage. However, the proportion of patients, who are metastatic at the time of diagnosis or who develop metastasis as the disease progresses, remains significant. In these cases, the range of available therapeutic options is more limited, and although recent therapies are increasingly effective, at this stage we can no longer generally speak of a complete cure, and it is understandable that the chances of survival are significantly lower, especially in metastatic disease.

There are several options for pharmacotherapy of PCa. One is androgen deprivation therapy (ADT). The main goal of androgen deprivation (AD) is to reduce testosterone levels to the same level as castration. ADT therapy is standard therapy for distant metastatic disease, but can also be used for local tumors, especially as part of neoadjuvant, adjuvant, or combination therapy, in case of moderate to high risk disease, or disease spread to lymph nodes. More recently, 2nd generation hormone therapy (abiraterone, enzalutamide) has been integrated into PCa therapy. If the disease progresses with ADT hormone therapy, we speak of castration-resistant prostate carcinoma (CRPC). For CRPC, effective therapies that can in fact prolong patient survival are now available in a variety of ways. In this disease state, the aforementioned 2nd generation hormone therapy (also known as ARTA treatments) is increasingly used. In other cases, in addition to significantly reducing the symptoms of the disease, cytotoxic chemotherapy is a treatment option. Docetaxel is now the first choice in standard chemotherapy for metastatic castration-resistant disease. Docetaxel therapy increases overall survival by an average of approximately 2-3 months, and only a few patients experience long-term complete remission. The effectiveness of docetaxel therapy is limited by several factors. Undesirable side effects of the drug adversely affect the quality of life of patients (S.-E. Al-Batran et al., Ann Oncol., 2015, 26, 1244-1248; S. Tonyali et al., Curr Urol., 2016, 10, 169-173). Another major limiting factor in the use of PCa therapy is the frequent development of resistance to taxanes and other cytotoxic chemotherapy.

Thus, it can be concluded that the hormone-refractory PCa has become an important public health problem due to its aggressiveness, not always effective therapeutic options and high mortality rate. More recently, it has been recognized that its pathomechanism is characterized by an increasing presence of the neuroendocrine component, which explains, among other things, its androgen independence and resistance to chemotherapy. In light of these, it is particularly important task to understand the further details of the mechanism of action and to combat the problems that arise, on the one hand, in order to provide chemotherapy to patients for whom the therapy is expected to be truly effective (R R Gordon et al., PloS One, 2014, 9, e104271).

In this context, due to the still unmet needs for PCa drug therapy, great efforts are being made today to identify and develop a new, superior, effective therapeutic solution. The goal is that the use of the new formulation even in advanced disease allow patients to receive effective therapy for as long as possible.

One of such newer alternatives is the recent use of monoamine oxidase A (MAO-A) enzyme inhibitors for the treatment of PCa.

The Enzyme Monoamine Oxidase

The enzyme monoamine oxidase (MAO) belongs to the flavin-containing oxidase enzyme family (E.1.4.3.4); its two subtypes are known, MAO-A and MAO-B. The two subtypes are encoded by separate genes. The two subtypes are located in the outer membrane of the mitochondria, but their location and structure (with 70% amino acid sequence identity) are also different. The function of MAO is the oxidative deamination of endogenous and exogenous monoamines (primary, secondary and tertiary) in different organs (the two subtypes have different distributions and the levels of both increase with aging, but to different degrees). In the gross reaction, the aldehyde corresponding to the amine, ammonia and hydrogen peroxide are formed from the amine with one mole of oxygen. The latter can form additional reactive oxygen species (ROS) agents, including, by Fenton reaction, the particularly reactive hydroxyl radical. Thus, an MAO can contribute to oxidative stress and all its harmful consequences. The two subtypes have different substrate and inhibitor specificities, the substrates of MAO-A are serotonin and noradrenaline, while the substrates of MAO-B are benzylamine and 2-phenylethylamine, both isoform enzymes deaminate dopamine and tyramine (the latter substrate has a higher sensitivity to MAO subtype A).

Well-known, characterized selective inhibitors are also used in drug therapy. However, ‘selective’ inhibitors also lose their selectivity at higher doses (e.g., Z. Fisar et al., Biogenic Amines, 2011, 25, 59-81) which demonstrates that the selectivity is only relative to MAO-A and MAO-B. inhibitors (i.e., a ‘selective’ MAO-A inhibitor always has MAO-B inhibitory activity depending on the concentration, and vice versa).

Inhibitors of both subtypes have long been used as drugs. The MAO-A inhibitor phenelzine (irreversible) and tranylcypromine (irreversible) are marketed as antidepressants, while the selective MAO-B inhibitor selegiline and rasagiline have long been proven in the treatment of Parkinson's disease (PD) and more recently safinamide is also used. The side effect profile of MAO-B inhibitors is favorable: they are generally well tolerated, with few undesirable side effects observed (B. J. Robottom, Patient Preferred Adherence, 2011, 5, 57-64).

Another known (although not used in drug product) selective MAO-A inhibitor is clorgylin.

The Role of Monoamine Oxidase-A Enzyme in Prostate Carcinoma

Since the turn of the millennium, it has been reported in several publications that the enzyme MAO-A may play a role in the development of prostate carcinoma as well as in resistance to the chemotherapeutic agent used to treat PCa.

In one pioneering work (L. True et al., Proc Natl Acad Sci USA, 2006, 103, 10991-10996), a correlation was sought between the Gleason scale classification of PCa tissue samples and their gene expression profile. It was found that carcinoma with a higher Gleason rating and the MAO-A expression are interrelated, which may also interpret the previous clinical finding that prostate tumor aggression and therapy resistance correlate with the neuroendocrine component signalling the disease progression. Not long after, the impressive results of another working group also supported the involvement of MAO-A in PCa (D. M. Peehl et al., J. Urol. 2008, 180, 2206-2211). In subsequent publications, several working groups reported the inhibitory effect of MAO-A inhibitors observed in various human PCa cell lines. More recent studies, in addition to elucidating the mechanism of tumor growth, have also paid close attention to the conditions under which resistance develops. A clinical trial involving patients with prostate cancer treated with docetaxel and mitoxantrone (NCT00017563; T. M. Beer et al., Clin. Cancer Res. 2004, 10, 1306-11) and the subsequent recognition provided one of the most interesting results. Gene expression changes in the prostate as a result of treatment have suggested that increased MAO-A expression may be responsible for resistance to docetaxel (R. R. Gordon, et al., PLoS One. 2014, 9, e104271). Another paper also analyzed the molecular mechanism of MAO-A-dependent resistance (J B Wu et al., J. Clin. Invest. 2014, 124, 2891-2908): among others activation of VEGF and its co-receptor and reactive oxygen species (ROS) play role in the pathomechanism (the latter induce resistance through their oxidative stress-inducing effect). In particular, the ROS pathomechanism element also supports that the predominantly enzyme function of the MAO-A protein is related to resistance. These clinical experiences—high MAO-A expression closely correlated with poorer clinical status in PCa patients—also confirmed that MAO-A inhibitors may have therapeutic significance in the treatment of PCa. And another preclinical study validates this suggestion. It has been found that the growth and proliferation of both androgen-sensitive and castration-resistant human prostate cells are inhibited by certain MAO-A inhibitors (clorgylin and phenelzine), in particular clorgylin significantly reduced the growth of enzalutamide-resistant cells. They also come to this conclusion in another study in which suppression of MAO-A function in epithelial cells in prostate adenoma carcinoma reduced both prostate size and the incidence of invasive carcinoma (C.-P. Liao, Oncogene, 2018, 37, 5175-5190).

Based on the foregoing, MAO-A inhibitors, alone or in combination, may be useful in the treatment of patients with advanced prostate cancer. However, in the case of MAO-B selective selegiline, due to its more modest effect on PCa cell line (based on in vitro experiments), its use for the treatment of PCa has not been suggested for a long time. This is indicated by the discussion of a paper published in 2019 (S. Gaur et al., Prostate, 2019, 79, 667-677) discussing the effect of MAO-A inhibitors against PCa (see the title: ‘Effect of Monoamine oxidase A (MAO-A) inhibitors on androgen-sensitive and castration-resistant prostate cancer cells’), and the weaker effect of selegiline is considered to be due to the weak MAO-A activity coming from the selectivity to MAO-B. That is, the use of MAO-B inhibitors for the treatment of PCa is not discussed in this article either, on the contrary, due to their weak MAO-A inhibitory effect, the article teaches away from the use of MAO-B inhibitors for this purpose.

Based on the role of the MAO-A enzyme in PCa outlined above, only a single MAO-A inhibitor has so far been initiated in a clinical trial. The MAO-A irreversible inhibitor phenelzine (which is used to treat depression in the US, but due to its side effects to a limited extent; phenelzine inhibits the MAO-A subtype significantly more strongly than-B) is currently being used in patients with two phase II clinical trials study in the US; phenelzine is used alone in one case and in combination with docetaxel in the other.

Monoamine Oxidase-B Enzyme and Prostate Carcinoma in the Literature

Although there are sources in the literature (see below) that the inhibitory effect on MAO-B and PCa may be coupled, upon detailed study, it becomes clear that what has been described does not refute the findings in the above-mentioned S. Gaur et al. (Prostate, 2019, 79, 667-677) that i) MAO-B inhibitors are not effective against PCa (in this article, MAO-B selective selegiline was investigated and was not found to be suitable for the treatment of PCa); ii) the mild effect observed is due to the weaker but existing MAO-A activity of the MAO-B inhibitor molecule.

In Sharma et al. (Bioactive Dimeric Acylphloroglucinols from the Mexican Fern Elaphoglossum pateaceum, J. Nat. Prod., 2019, 82, 785-791, Mar. 28, 2019, abstract and Tables 2 and 4, see page 788) two prenylated acylfluoroglucinol compounds isolated from Elaphoglossum paleacum (“prenylated acylphloroglucinol”; compounds 1 and 2) were tested. According to the measurements, the plant extracts have a mixed MAO-A and MAO-B inhibitory effect depending on the solvent used for the extraction: for the hexane solvent 25.0% MAO-A inhibitory effect and 42.5% MAO-B inhibitory effect was measured, while these values were 26.5% and 23.7% respectively, for the extract obtained with chloroform. Compound 1 obtained by the purification process can be considered as a somewhat selective MAO-B inhibitor according to Table 2. The compounds have been tested on a variety of tumor cell lines, including PCa cell lines. According to Table 3, for the PC3 human PCa cell line, Compound 1 is more potent than Compound 2. However, the skilled person does not conclude from this result that the anti-PCa effect is due to the MAO-B inhibitory effect, because one of the serious shortcomings of the studies is that no known effective MAO A and MAO B inhibitor references were used in the experiments on the prostate carcinoma cell line (PC3), which are essential for the validation of the model and thus for a well-established evaluation of the efficacy of the test substance in the context of MAO inhibition. Therefore, the inhibitory effect of the disclosed compounds 1 and 2 on the PCa tumor cell line is not at all necessary to be related to their already weak MAO enzyme inhibitory activity. It is well known that cell lines are only suitable for a relative classification of an effect within one series, using relevant references, because the properties (sensitivity) of the model vary from case to case. Furthermore, it is known that only a small amount of MAO protein, especially MAO B protein, is present in the PC3 cell line, so to measure MAO B inhibitors, the model should be validated with known high selectivity MAO B inhibitors if, on the basis of the results, one wishes to refute the above finding of the scientifically more credible S. Gaur et al. publication.

Furthermore, one skilled in the art could reasonably assume that the effect of the two compounds on the PC3 cell line was not due to MAO inhibition but to other types of cytotoxic effects.

It is also an important fact that the authors themselves do not either suggest a correlation between the effect on the PCa cell line and the MAO-B inhibitory effect. Based on all this, the published results do not provide any guidance to the person skilled in the art to refute the results according to S. Gaur et al. article, i.e. that the use of MAO B inhibitors in the treatment of prostate carcinoma is more promising than that of MAO-A inhibitors.

One of the many studies in U.S. Patent Application No. 2018/0185303 A1 addresses the effect of MAO-B, namely, where the presence of protein components was examined in a sample of prostate tissue from 88 patients with prostate carcinoma [0051]. On this basis, the following is established in connection with MAO proteins (see also paragraphs [0036] and [0041]):

i) The epithelial level of MAO-A protein is high, so it may play a role in the pathomechanism of prostate carcinoma, thus MAO-A inhibitors may play a role in the therapy of prostate carcinoma. This statement of the specification is supported by MAO-A (and non-MAO-B!) inhibitors, relevant experimental data from publications 6-8 also referred by the specification, and additional in vitro and in vivo data described in the specification.

ii) The role of MAO-B protein (its level determined by immunohistochemistry in the above prostate tumor patients, FIG. 7B) is discussed in connection with its presence in the stroma, so it may be a target in the treatment of prostate carcinoma. In addition, the description does not disclose any experiment/data that would explain the role of the MAO-B protein or its inhibitors. In contrast, the remainder of the description examines the role of MAO-A proteins and their inhibitors in great detail.

It follows from the above description that an MAO-B inhibitor (such as selegiline, see) [0041]) would be effective in prostate carcinoma by ‘targeting’ MAO-B activity in stromal cells. In paragraph [0041], it is emphasized that selegiline, an MAO-B inhibitor, should be used as a secondary agent only in addition to the primary agent clorgylin, which targets epithelial MAO-A. The secondary role of the MAO-B effect according to the cited description is also supported by the fact that the description does not provide experimental data on the MAO-B inhibitory effect and it is not assumed that a MAO-B inhibitor can have a positive effect on PCa also in epithelial cells.

Thus, it can be deduced from the cited description that an MAO-B inhibitor can be effective in a carcinoma model containing only stromal elements, since epithelial cells are virtually free of MAO-B (see paragraph 0051, lines 7-9 of this specification). Furthermore, the use of MAO-A inhibitor is described to be essential as the MAO-A protein has high epithelial level.

In contrast, our studies suggest that the anti-prostate carcinoma effect of the MAO-B inhibitor selegiline does not require the presence of stromal cells, and the anti-prostate carcinoma effect does not require the administration of a MAO-A inhibitor. This is because our studies have demonstrated that the MAO-B inhibitor used is effective on the PC3 human prostate carcinoma cell line alone (the study was performed in an accepted model of prostate carcinoma, see the examples). However, the PC3 cell line does not contain stromal elements, hence the presence of stromal elements is not required for the MAO-B inhibitory compound (selegiline). The positive results obtained confirm that the MAO-B inhibitors tested are also suitable for the treatment and/or prevention of PCa also in epithelial cells.

The fact of the absence of stromal elements in the PC3 cell line also follows from that all cells in the PC3 cell line are aneuploid [Kaighn M E, Narayan K S, Ohnuki Y, Lechner J F, Jones L W., Establishment and characterization of the human prostatic carcinoma cell line (PC-3). Invest Urol. 1979 July; 17 (1): 16-23 and Yasushi Ohnuki, Maureen M. Marnell, Merrill S. Babcock, John F. Lechner, and M. Edward Kaighn., Chromosomal Analysis of Human Prostatic Adenocarcinoma Cell Lines, CANCER RESEARCH 40, 524-534, March 1980].

Note that aneuploidy (abnormal chromosome number) is a definite property of tumor cells. 90% of solid tumors are aneuploid [Taylor, A. M. et al. Genomic and functional approaches to understanding cancer aneuploidy; Cancer Cell 33, 676-689.e3 (2018)]. Furthermore, Olumi et al. [Aria F. Olumi, 2 Gary D. Grossfeld, 2 Simon W. Hayward, Peter R. Carroll, Thea D. Tlsty, 3 and Gerald R. Cunha; Carcinoma-associated Fibroblasts Direct Tumor Progression of Initiated Human Prostatic Epithelium; CANCER RESEARCH 59, 5002-5011, Oct. 1, 1999] prepared from both normal (NHPF) and tumor human prostate tissue (CAF) stromal fibroblast primary cell cultures, and karyotype analysis of the cells revealed that both normal and tumor tissue stromal fibroblasts had a normal diploid karyotype. Because all cells in the PC3 cell line are aneuploid, tumor-derived stromal fibroblasts, in turn, are diploid, it may be established that the PC3 cell line does not contain stromal elements.

Based on the above, it can be said that there is no known method or suggestion for PCa treatment in the literature where an MAO-B inhibitor alone is recommended for use. Based on the direct and indirect preclinical data described above, one skilled in the art will conclude that of the two subtypes of the MAO enzyme, the MAO-A subtype plays a major and unavoidable role in PCa tumorigenesis and resistance to chemotherapy. Each of the works cited above is based on the known fact that of the two MAO subtypes, the MAO-A subtype is dominant in the prostate, the expression of which is increased in PCa. Thus, it is understood that the MAO-A subtype has been given a prominent role so far, while the possible role of the MAO-B subtype in PCa is mentioned in the literature at most secondarily, only in connection with stromal tissues.

Discovery According to the Invention

Surprisingly, we found that the MAO-B enzyme has a much more significant role in the pathomechanism of PCa than can be inferred from the ratio of MAO-A and MAO-B subtypes obtained in PCa cells. In our experiments, we examined two irreversible MAO-B inhibitors in vitro and in vivo in a PCa model. Surprisingly, we found that selective MAO-B inhibitors, which inhibit the MAO-B subtype from the two subtypes of the monoamine oxidase enzyme, have a protective effect on their own, i.e., without the use of selective MAO-A inhibitors in parallel. According to our experiments on the PCa cell line (see Example 1), this is especially true for selegiline, but rasagiline also has a significant protective effect (see also Example 1).

In particular, these studies demonstrated that the selective MAO-B inhibitor selegiline and rasagiline also alone significantly reduced tumor cell viability (core viability and proliferation rate) in both LNCaP and PC3 cell lines, which are considered as hormone-sensitive and hormone-insensitive in vitro models of PCa. It can be seen that the selective MAO-B inhibitors exert their effects at a slightly higher concentration than the selective MAO-A inhibitor clorgylin used as a reference standard, but achieve the efficacy of clorgylin in their efficacy. The results of our in vivo experiments in a human PC3 xenograft model of NSG SCID mice, considered as an in vivo model of PCa, also demonstrated that selegiline, like clorgylin, significantly reduced the rate of tumor growth (see Example 2).

It should be emphasized that our animal experiments show that the PCa inhibitory effect of MAO-B inhibitors is already present in a dose that is safe and does not cause undesirable side effects in the treated organisms. Our experiments demonstrate that selegiline and rasagiline alone are suitable for the therapeutic treatment of PCa. Since these are two representative members of MAO-B inhibitors, we can reasonably assume that the observed positive effect also occurs with other MAO-B inhibitors.

It is further noted that the MAO-B inhibitor compound (preferably selegiline and/or rasagiline) may be used in combination with one or more other agents in the use according to the present invention or in the pharmaceutical compositions prepared thereby, wherein the other agent is preferably anticancer agent, and more preferably is for the treatment of PCa in clinical practice.

### BRIEF DESCRIPTION OF THE INVENTION

The Invention Relates to:

1. A selective monoamine oxidase-B (MAO-B) inhibitor compound for use in the prevention or treatment of prostate carcinoma (PCa), wherein no selective MAO-A inhibitor compound is co-administered during use.

2. A selective MAO-B inhibitor compound for use according to item 1, wherein the MAO-B inhibitor compound is selected from the group consisting of selegiline and rasagiline.

3. A selective MAO-B inhibitor compound for use according to item 1 or 2, wherein the MAO-B inhibitor compound is selegiline.

4. A selective MAO-B inhibitor compound for use according to any one of items 1-3, wherein other agents for the treatment of PCa are co-administered and/or radiotherapy is used in conjunction with or alternately with the selective MAO-B inhibitor compound.

In the above application, co-administration also includes the case where the selective MAO-B inhibitor selective compound is administered continuously, while the other active ingredient is optionally administered intermittently (e.g., with intervals of several days/weeks). It is understood that radiotherapy for localized PCa is also performed continuously, while in case of the diagnosis of metastatic PCa it is performed intermittently with the addition of the MAO-B selective compound and/or the other drug as described above.

The above active ingredients may be co-administered separately (e.g. as separate tablets, solutions, the latter in the form of an infusion or injection) or in a single formulation (in a mixed tablet, in a solution containing the active ingredients together (as an infusion or injection)). The various active ingredients to be used together may also be presented in the form of a kit adapted to the dosing regimen, wherein the kit has its active ingredients formulated separately in the same dosage unit, optionally in a different formulation type (e.g. tablet and injection or lyophilized powder).

5. A selective MAO-B inhibitor compound for use according to item 4, wherein the (as appropriate: one or more) other active ingredient for the treatment of PCa is selected from the group consisting of taxane derivatives acting on the microtubule system, preferably docetaxel or cabazitaxel (optionally in combination with a steroid); platinum preparations, preferably carboplatin (optionally in combination with a non-steroidal anti-inflammatory drug); topoisomerase inhibitors (optionally in combination with a non-steroidal anti-inflammatory drug); antitumor compositions with a complex mechanism of action, such as mitoxantrone (optionally in combination with a non-steroidal anti-inflammatory drug); hormone therapeutic agents such as abiraterone or enzalutamide; androgen deprivation agents; androgen receptor agents; kinase inhibitors; antiangiogenesis agents; immunotherapeutic preparations; anti-inflammatory drugs; biological preparations having anticancer effects, anticancer preparations made from natural substances, e.g. anticancer preparations made from herbs; and compositions for inhibiting bone metastasis.

6. A selective MAO-B inhibitor compound for use according to item 5, wherein the other agent for treating PCa is a taxane derivative, preferably docetaxel.

7. A selective MAO-B inhibitor compound for use according to any one of the preceding items, wherein the PCa is castration-resistant prostate carcinoma (CRPC).

8. Use of a selective MAO-B inhibitor compound in the manufacture of a medicament for the treatment of PCa.

Also in the Case of the Invention According to Item 8 Above the Features Disclosed According to Items 2 to 7 Above Constitute Preferred Sub-Cases.

9. A method of preventing or treating prostate carcinoma (PCa) comprising administering to a human or animal in need thereof a selective MAO-B inhibitor compound in a pharmaceutically effective amount without administering a selective MAO-A inhibitor compound.

10. The method according to item 9, wherein the selective MAO-B inhibitor compound is co-administered or alternately administered with other agents for the treatment of PCa and/or radiation therapy is also used.

Also in the Case of the Invention According to Items 9 and 10 Above the Features Disclosed According to Items 2 to 8 Above Constitute Preferred Sub-Cases.

The invention is applicable to mammals, especially humans, but also to animals (e.g. domestic animals such as dogs, cats) where the positive effect also appears. In animals, it may be advantageous to use platinum preparations and/or topoisomerase inhibitors and/or antitumor preparations with complex mode of action, optionally in combination with a steroid and/or non-steroidal anti-inflammatory drug (NSAID), where the administration of the non-steroidal anti-inflammatory drug (NSAID) is preferred.

### DETAILED DESCRIPTION OF THE INVENTION

The term “MAO-B inhibitor compound” includes salts (preferably HCl or sulfate salt), hydrates, and any isomers or mixtures thereof of the compound in question. Furthermore, a “MAO-B inhibitor compound” refers to an active ingredient that, at a concentration that exerts significant MAO-B inhibition, preferably only slightly or negligibly inhibits the MAO-A enzyme, i.e., is a selective MAO-B inhibitor. The “MAO-B inhibitor compound” is preferably selected from selegiline, rasagiline and safinamide, where selegiline and rasagiline being preferred, and selegiline (also known as (−)-deprenyl) being particularly preferred.

The dosage form of the pharmaceutical compositions of the present invention (“compositions” in short) is not critical, thus they may be administered oral, intravenous, intramuscular, parabulbar, retrobulbar way, in the form of subtenon, intracameral, intravitreal and other injections, but may be administered sublingually or transdermally.

The composition can be administered in solid, semi-solid and liquid forms. Suitable liquid forms include, but are not limited to, solutions, tinctures, syrups, emulsions and suspensions.

In addition to the above, the pharmaceutical compositions of the present invention may contain one or more pharmaceutical excipients (e.g. processing aids, carriers, surfactants, colorants, sweeteners, solvents, suspending agents, coatings etc.). Controlled release formulations and organ-specific delivery formulations are preferred.

The above-mentioned pharmaceutical compositions are prepared by mixing the preferred selegiline or rasagiline or their salts (preferably, for example, the hydrochloride or methanesulfonate salt) and one or more excipients, and then converting the resulting mixture into a pharmaceutical composition in a manner known per se, including nanotechnology-based solutions. Applicable methods are known in the literature, such as Remington's Pharmaceutical Sciences.

The pharmaceutical compositions according to the present invention will generally contain a unit dose. The actual dose depends on a number of factors and is determined by the physician on the basis of standard parameters known in the art (body weight, body surface area, age, stage and severity of the disease etc.).

Selegiline or rasagiline, preferably in the form of a salt thereof, preferably the hydrochloride or methanesulfonate, may be used in the pharmaceutical compositions prepared according to the use according to the present invention, optionally in combination with one or more other active ingredients and/or radiation therapy. Those drug combinations are considered as preferred other drug combinations which are used in the treatment of PCa, in which the mechanism of action of each drug component is different. In these combinations, chemotherapeutic and/or hormone therapeutic agents are preferred. Preferred other agents are taxane derivatives acting on the microtubule system, preferably docetaxel or cabazitaxel (optionally in combination with a steroid), particularly preferably docetaxel, hormone therapeutic agents such as abiraterone or enzalutamide. The active compounds according to the invention and the compositions containing them can also be used in combination with other chemotherapeutic and/or hormone-therapeutic agent(s), depending on the patient's condition and disease, said agent(s) being selected for example from the following group: androgen deprivation agents, androgen receptor agents, kinase inhibitors, antiangiogenesis agents, immunotherapeutic preparations, biological preparations with anti-cancer activity, anticancer preparations made from natural substances, e.g. herbal anticancer preparations, bone metastasis inhibitors and/or MAO-A inhibitors.

In combination formulations, the two (or possibly more) active ingredients may be formulated together (in a single dose), but it may also be advantageous for each active ingredient [or subgroup(s) thereof] to be formulated separately. Such a separately formulated formulation also allows the co-administration of the active ingredients or, where appropriate, the individual active ingredients [or subgroup(s) thereof] to be administered in a time-shifted manner to the human or animal in need thereof.

In practice, it is also important to administer one active ingredient (preferably the MAO-B inhibitor) continuously during treatment, while the other active ingredient is administered intermittently (using an effective dose of the active ingredient in each case). The examination protocol for this type of combination treatment is detailed in Example 3. Clinical trials will be used to investigate the beneficial effects of the concomitant use of selegiline and docetaxel.

Notwithstanding the above assumption, the use of combination formulations (where it is preferred that the active ingredients act by a different mechanism of action) may be considered conventional in the art, so that the present invention also relates to applications and formulations wherein MAO-B the inhibitory compound is present together with another (preferably chemotherapeutic and/or hormone therapeutic) active ingredient.

The use of the invention encompasses use in the treatment of humans and animals with a prostate. As mentioned in the introduction, PCa is also a relevant disease in a number of animals.

### BRIEF DESCRIPTION OF THE DRAWINGS

FIG. 1: Validation of cell viability assays. The absorbance and relative luminescence values are directly proportional to the cell number (confirmed by the value of the R2 coefficient of determination). A: MTS method, 60 min incubation. B: MTS method, 120 min incubation. C: CellTiter-Glo luminescent cell viability assay.

FIG. 2: Effect of clorgylin, rasagiline and selegiline on LNCaP cell viability. A: results of the MTS method after 48 hours of clorgylin treatment. Statistical analysis: unpaired t-test (p<0.05) and Kruskal-Wallis one-way ANOVA, Dunn's post hoc test (p<0.05) B: results of CellTiter-Glo viability method after 48 hours of clorgylin treatment. Statistical analysis: unpaired t-test (p<0.05) and one-way ANOVA, Bonferroni post hoc test (p<0.05). C: results of the CellTiter-Glo viability method after 48 hours of rasagiline treatment. Statistical analysis: Kruskal-Wallis one-way ANOVA, Dunn's test (p<0.05). D: results of CellTiter-Glo viability method after 48 hours of selegiline treatment. Statistical analysis: Kruskal-Wallis one-way ANOVA, Dunn's test (p<0.05).

FIG. 3: Effect of clorgylin, rasagiline and selegiline on PC3 cell viability. Results obtained with the CellTiter-Glo viability method after 48 hours of treatment. Statistical analysis: one-way ANOVA, Bonferroni post hoc test (p<0.05). A: results of clorgylin treatment. B: results of rasagiline treatment. C: results of selegiline treatment.

FIG. 4: Tumor growth rate as a function of time in NSG SCID mouse human PC3 xenograft model. From the 14th day of the experiment, the animals received the following treatments: phys. salt: physiological saline, C10: clorgylin 10 mg/kg dose, S10: selegiline 10 mg/kg dose. Statistical analysis: one-way ANOVA, Fisher post hoc test (p<0.05).

FIG. 5: The results shown in the bar graph show that selegiline, docetaxel, and combinations thereof reduce PC3 cell viability in a concentration-dependent manner. CellTiter-Glo viability assay results after 48 hours of treatment.

FIG. 6: The graph shows how prostate volume decreased in the treated animal during treatment time.

The following experimental examples illustrate the invention but are not intended to be limiting.

### EXAMPLES

### Example 1. Investigation of the Effect of the Selective MAO-B Inhibitor Rasagiline and Selegiline and the Selective MAO-A Inhibitor Clorgylin in In Vitro Models of Human Prostate Carcinoma: Investigation of the Effect on the Viability (Viability and Proliferation Rate) of LNCaP and PC3 Cells

This example describes the study of rasagiline and selegiline and the effect of clorgylin as a reference standard on cell lines accepted as an in vitro model of prostate carcinoma (hormone-sensitive: LNCaP and hormone-insensitive: PC3).

In the experiments, the active ingredients were used in the form of salts commonly used in pharmaceutical therapy, such as selegiline and clorgylin hydrochloride salt and rasagiline in the form of methanesulfonate (mesylate) salt.

Cell Lines

LNCaP and PC3 cell lines were obtained from the European partner of the ATCC (American Type Culture Collections), LGC Standards (Wesel, Germany). Cell lines were maintained and treated according to the protocol of Gordon et al. (PloS One, 2014, 9, e104271), with some modifications, as follows. The PC3 cell line does not contain stromal elements as stated by the manufacturer.

RPMI-1640 (Lonza) medium with 10% heat-inactivated FBS (fetal bovine serum, Sigma) and 100 U/ml Penicillin/Streptomycin was used to grow the cells. Cells were treated with drugs freshly dissolved in RPMI medium. Treatment concentrations were determined by widening the concentration range. After 48 h of treatment, cell viability was also quantified using the MTS reduction assay (Promega) as well as a CellTiter-Glo kit (Promega) based on ATP level measurements.

Viability Studies

In the MTS reduction method (CellTiter 96® AQueous One Solution Cell Proliferation Assay, Promega Corp, Madison, Wash.), 20 microliters of MTS reagent was used per sample according to the manufacturer's protocol, and after 120 minutes of incubation, absorbance was measured at 492 nm with a microplate reader.

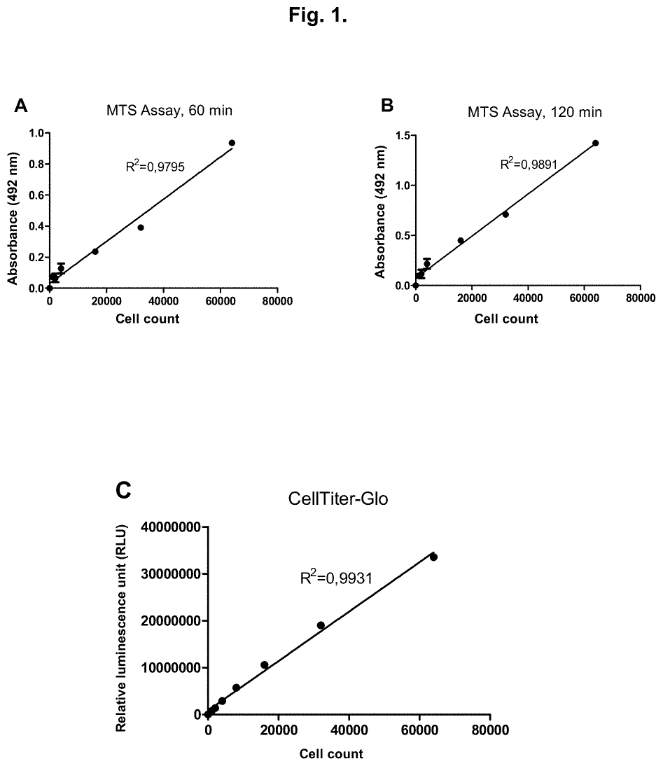

In the studies, we modified the protocol of Gordon et al (2014) as follows:
A volume of 20 microliters per well was used from the MTS reagent as recommended by the manufacturer (10 microliters were used by Grodon et al.).
In our case, an incubation time of 120 min was used to increase accuracy (Gordon et al incubated for 60 min). Note that the manufacturer does not give explicit instructions regarding the incubation time, so it was considered necessary to validate the accuracy of the absorbance values obtained after 60 and 120 minutes of incubation, respectively (FIGS. 1/A and 1/B, respectively). During validation, measurements were performed with a given number of cells per well, and absorbance values were plotted as a function of cell number. A calibration line was placed on the data points by linear regression analysis and the R² coefficient of determination was determined. R² is a number between 0 and 1, which shows the extent to which the cell number affects the absorbance value (if R²=1, then 100%, if R²=0 it does not affect it at all). In our study, based on the R² value of the line, we found that the relationship between absorbance values and living cell number is directly proportional after both 60 and 120 minutes of incubation, however, more accurate results are obtained with 120 minutes of incubation than with 60 minutes of incubation time.
Absorbance values were measured at 492 nm (Gordon et al. 450 nm filter was used). According to the manufacturer's description, the wavelength of the absorption maximum of formazan resulting from the reduction of MTS is 490 nm, so it is recommended to perform the measurement at this wavelength. According to the manufacturer's recommendation, the absorbance measurement can be performed in the wavelength range of 440-550 nm.

The CellTiter-Glo assay based on ATP level measurements was performed according to the manufacturer's protocol of pipetting 100 microliters of Celliter-Glo reagent into wells containing 100 microliters of medium, then shaking for 2 minutes and incubating for another 10 minutes. The luminescence signal was then detected with a PerkinElmer AlphaLisa instrument.

To support the applicability of the CellTiter-Glo viability assay method, we validated the method on a PC3 cell line. In this procedure, the measurement was performed with a given number of living cells per well, and the relative luminescence values obtained were plotted as a function of the cell number. A calibration line was placed on the data points by linear regression analysis, and based on the R2 value, it was determined that the relative luminescence value obtained during the measurement was directly proportional to the number of living cells (FIG. 1/C).

Statistical Analysis

GraphPad Prism 8 software was used for statistical analysis of the data. To validate the MTS method and the CellTiter-Glo viability assay, linear regression analysis was used, in which the absorbance (492 nm) and relative luminescence values were plotted as a function of cell number, and the R2 value was determined.

To reproduce the literature data, we also used the unpaired t-test also used by Gordon et al (2014) to compare control and treated groups. However, in our statistical analyzes, we found that the unpaired t-test used in the literature is not an appropriate statistical method for the experimental set-up used (control and multiple parallel treatments with different concentrations), and the one-way ANOVA method should be used instead. For some results (LNCaP chlororlin MTS method and LNCaP rasagiline and selegiline CellTiter-Glo method) Kruskal-Wallis non-parametric one-way ANOVA method was used with Dunn test, while in other cases one-way ANOVA method was used with Bonferroni post hoc test.

In our experiments, we performed three parallel experiments, and the experiments were repeated to increase the number of items. For the statistical analysis of two separate experiments the results were pooled, however, the absolute values obtained during the luminescence measurements cannot be used to combine the results. This is because the CellTiter-Glo reagent loses its activity during storage and possible thaw-freeze cycles. Thus, a CellTiter-Glo reagent from a single source can give different relative luminescence values (RLUs) at the same cell number in two different experiments performed at different times. However, in one experiment, the RLU reliably gives the cell number, and accordingly, the percentage viability value of the treated condition relative to the control RLU can be determined with certainty; values from different experimental series can be combined.

Combined results from two separate experiments were used to calculate relative viability values.

During the evaluation, we normalized to the mean of the control of the given experiment, so the mean of the control was 100% and the percentage of each individual data point relative to the mean of the control was calculated accordingly. One-way ANOVA with Bonferroni post hoc test with p<0.05 significance level was used for statistical validation of viability changes as described above.

Results

Viability Results in LNCaP Cell Line

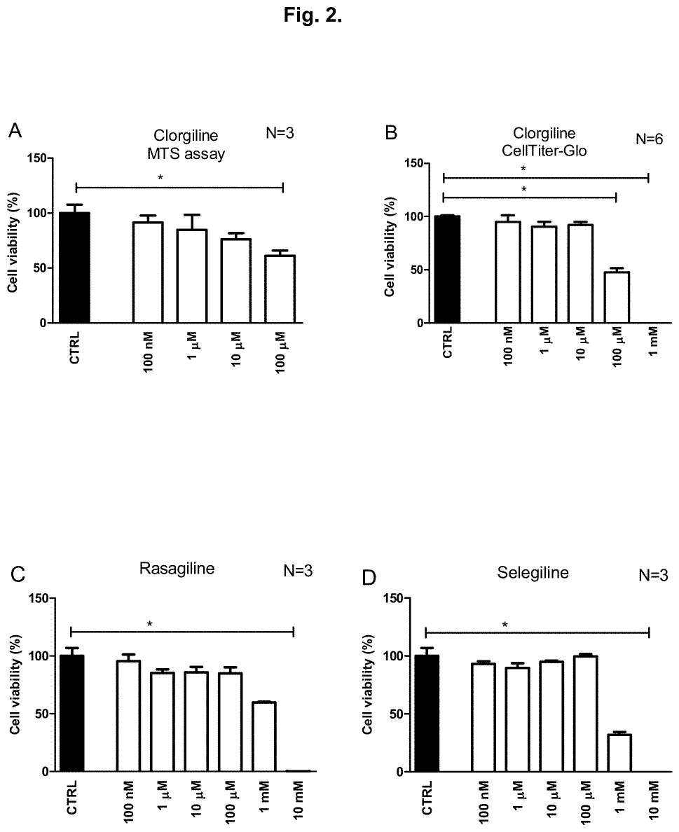

The experiment was validated by measuring the effect of chlorine used as standard on the viability of LNCaP cells. Our results measured by the MTS method showed a good agreement with the data in the literature (R. R. Gordon et al., PloS One, 2014, 9, e104271): it can be concluded that clorgylin reduces the viability of LNCaP cells in a concentration-dependent manner; in our experimental setup, we obtained a significant viability-reducing effect at a slightly higher concentration compared to the literature. Based on statistical analysis, treatment at a concentration of 100 micromolar significantly reduced the viability of LNCaP cells after 48 h of treatment (FIG. 2/A). The other method we used, the CellTiter-Glo viability test based on the measurement of ATP level, also gave results similar to the MTS method (FIG. 2/B).

Further, we used ATP level measurement in our studies. It is known that the viability method based on ATP level measurement provides more accurate and reliable results than the MTS method, another advantage of it is that it can be reliably standardized. In our experiments, both rasagiline and selegiline significantly reduced the viability of LNCaP cells. A significant viability-reducing effect was observed for both drugs at 10 mM after 48 hours of treatment; however, this trend is also clearly observed for the 1 mM treatment (FIGS. 2/C and 2/D).

In this study, the selective MAO-B inhibitor rasagiline and selegiline, as well as the selective MAO-A inhibitor clorgylin used as standard, significantly reduced the viability of LNCaP cells in a concentration-dependent manner.

Viability Results in PC3 Cell Line

The selective MAO-B inhibitor rasagiline and selegiline as well as the selective MAO-A clorgylin were also tested in a PC3 cell line. The PC3 cell line, in contrast to the LNCaP cell line, is hormone-insensitive, i.e. it does not respond to hormone therapy, and is a model of the more aggressive tumor type, which is essentially incurable according to our current possibilities.

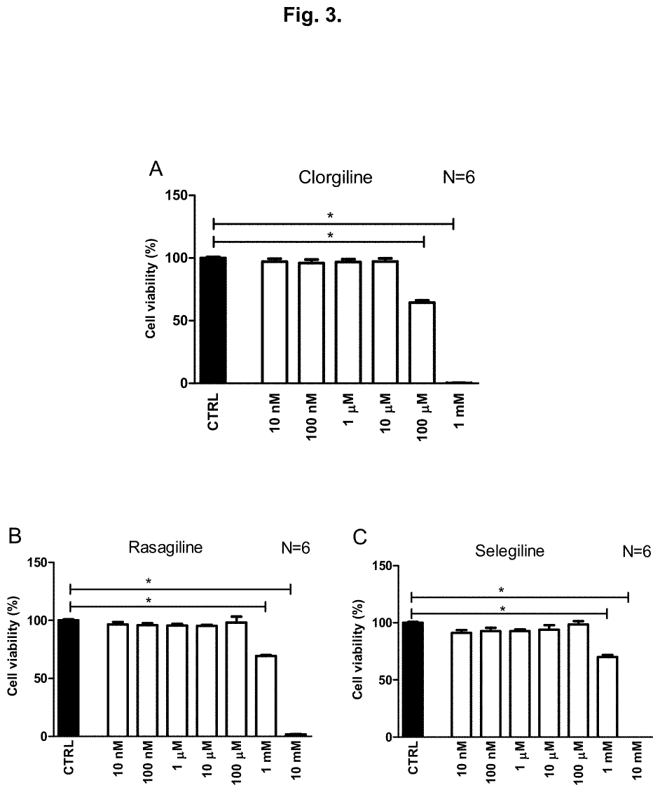

As indicated by the viability data based on ATP level measurements, clorgylin used as standard at 100 micromolar and 1 mM concentrations significantly reduced the viability of PC3 cells after 48 h (FIG. 3/A).

According to our experiment, the selective MAO-B inhibitor rasagiline and selegiline, like the selective MAO-A inhibitor clorgylin, showed a significant viability-reducing effect at 1 mM and 10 mM concentrations after 48 h (FIGS. 3/B and 3/C).

In this study, the selective MAO-B inhibitor rasagiline and selegiline, as well as the selective MAO-A inhibitor clorgylin used as standard, significantly reduced the viability of PC3 cells in a concentration-dependent manner.

### Example 2. Investigation of the Effect of the Selective MAO-B Inhibitor Selegiline and the Selective MAO-A Inhibitor Clorgylin in an In Vivo Model of Human Prostate Carcinoma: Investigation of the Effect on Tumor Growth in a Human Xenograft PCa Mouse Model

This example describes the study of the effect of selegiline as well as the effect of clorgylin as a reference standard on tumor growth in a human xenograft model in immunodeficient mice accepted as an in vivo model of PCa.

Animals

For our experiments, we used 25 3-month-old male NOD (Non Obese Diabetic) SCID (Severe Combined ImmunoDeficiency) Gamma mice (National Institute of Oncology Animal House). Immunodeficiency in these mice (Shultz et al, 1995) is associated with decreased T and B lymphocyte and NK cell function, as well as deficiency is developed in cytokine signaling pathways, in adaptive and innate immune systems. During the experiment, the animals were housed in the animal house of the János Szentágothai Research Center of the University of Pecs, and they were provided with standard rodent food and tap water ad libitum. The animals were kept at 20-24° C., 50-60% relative humidity, in a 12-12 hour dark-light cycle. The valid animal ethics permit (BA02/2000-54/2018) for the experiments was approved by the NEBIH on the proposal of the Committee on Animal Ethics at Work of the University of Pecs and the Scientific Ethics Council for Animal Experiments (ATET).

Culturing Tumor Cells, Preparing them for Injection

RPMI-1640 (Lonza) medium with 10% heat-inactivated FBS (Sigma) and 100 U/ml Penicillin/Streptomycin was used to proliferate the PC3 cell line (Wesel, Germany), which was also used for in vitro experiments. This cell line develops rapidly and with high reliability in NSG mice, making it much more suitable for in vivo studies than LNCaP cells; as mentioned above, due to its hormone insensitivity and aggressiveness, its use for testing active ingredients with an indication of significant therapeutic needs is particularly expedient. Adherent cells were detached from the growth surface with trypsin-EDTA solution (Sigma) and the cell suspension was washed three times with PBS (1000 RPM, 5 min). Cell counts were determined after trypan blue staining with a LUNA II automated cell counter (Logos Biosystems) and 50 million cells were resuspended in 5 ml PBS.

Tumor Cell Administration

After two weeks of acclimatization, on the first day of the experiment, the animals were deeply anesthetized with Na-pentobarbital (70 mg/kg) given intraperitoneally (i.p.), then the back hair was shaved and disinfected with 1-2 drops of 70% ethanol. Superficially, a 27G needle was inserted subcutaneously over the left thigh into the skin (5-10 mm) to which a 1 ml syringe containing cell suspension was attached. The suspension contained 1 million PC3 tumor cells in 100 microliters of PBS (0.1 M phosphate buffered saline, pH=7.4) and 100 microliters (2 mg/ml) of Matrigel (ECM Gel; Engelbreth-Holm-Swarm murine sarcoma, Sigma). Mice were individually housed until complete awakening and monitored for vital signs (J B Wu et al., J Clin Invest., 124, 2891-2908; C. Bastide et al., Prostate Cancer Prostatic Dis., 2002, 5, 311-315).

Measurement of Tumor Growth

On days 4, 7, 11, 14, 18, and 21 of the experiment, the length (a) and width (b) of the growing tumor were measured with a caliper, and then the tumor volume was calculated using the following formula (J B Wu et al., J Clin Invest., 124, 2891-2908):

V=a×b²/2 (mm³)

The general condition of the animals (hair, visible mucous membranes, basic neurological functions, locomotor activity, changes in body weight) was checked daily.

Treatment

The 25 mice used in the study were divided into 3 groups:

1) Solvent: physiological saline i.p. once daily (n=8)

2) C10: 10 mg/kg clorgylin i.p. once daily (n=7)

3) S10: 10 mg/kg selegiline i.p. once daily (n=10)

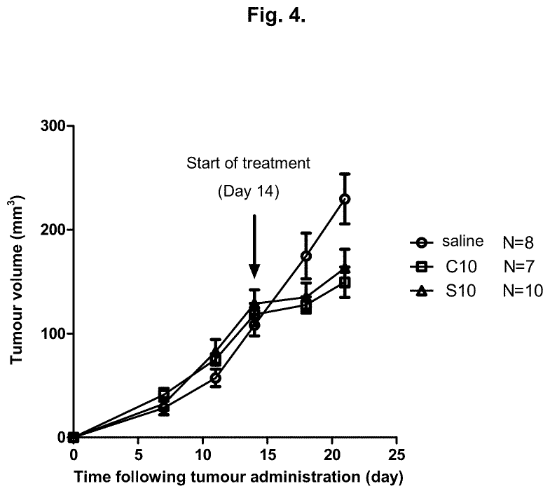

To examine the effect of active substances to be tested on tumor growth, as above, from day 14 onwards, mice were treated daily i.p. injection and the tumor size was continuously monitored. In our experiment, the saline-treated group served as a solvent control, while the selective MAO-A inhibitor clorgylin was used as a standard (J. B. Wu et al., J Clin Invest., 124, 2891-2908); in the experiment we examined the effect of the selective MAO-B inhibitor selegiline. The results are shown in FIG. 4.

Statistical Analysis

Tumor size comparisons were performed by one-way ANOVA followed by Fisher post hoc test. A normality test and analysis of variance homogeneity were also performed to examine the validity of the ANOVA.

Results

The results of the study are shown in FIG. 4.

It can be seen that in the human PC3 xenograft model of the NSG SCID mouse, selegiline (p=0.021) significantly reduced the rate of tumor growth compared to the group treated with physiological saline (day 21), similar to clorgylin (p=0.012).

### Example 3: Clinical Trial Protocol for the Efficacy and Safety of Drug Therapy with Docetaxel and Selegiline in Patients Suffering from Metastatic Castration-Resistant Prostate Adenocarcinoma

a) Brief description of the approved clinical protocol (OGYÉI protocol number: MAO201901, EudraCT (EU Clinical Trials Register) number: 2019-002685-12):

The aim of this study was to evaluate the efficacy and safety of selegiline+docetaxel therapy in patients with metastatic prostate adenocarcinoma. The study is performed in patients diagnosed with metastatic castration-resistant prostate adenocarcinoma whose clinical status requires using docetaxel therapy.

Brief Description of the Study

1. A Randomized, Docetaxel-Controlled Study

Study arm: selegiline+docetaxel treatment

Patients receive 10 mg selegiline therapy daily from the first day of docetaxel therapy.

Docetaxel therapy is administered every three weeks at a dose of 75 mg/m² (given as a single dose).

Docetaxel therapy is continued for up to 12 cycles.

Selegiline therapy can be used in addition to docetaxel therapy during progression.

Control arm: docetaxel treatment

Docetaxel therapy is administered every three weeks at a dose of 75 mg/m².

b) Description of Specific Clinical Trials and their Results

In this experiment, the effect of concomitant use of docetaxel and selegiline on the viability of a PC3 prostate carcinoma cell line was investigated.

Material and Method:

In our studies, we used a hormone-sensitive PC3 prostate carcinoma cell line. The culture of the cell line and the study of the effect of the combination of docetaxel and selegiline were performed as previously described, with the changes detailed below.

Selegiline, docetaxel, and selegiline+docetaxel combination studies were performed in parallel, simultaneously, with the same reagents, on a cell population from the same cell culture. For the selegiline, docetaxel and selegiline+docetaxel combination studies, the following changes were applied from the protocol according to the examples of the application:
Treatments with selegiline alone were performed at concentrations ranging from 250 μM to 1 mM (250 μM, 500 μM, 750 μM, 1 mM), while docetaxel alone was administered at a concentration of 1 μM. For combination treatments, 1 μM docetaxel was added to the concentrations corresponding to the selegiline treatments (250 μM selegiline+1 μM docetaxel, 500 μM selegiline+1 μM docetaxel, 750 μM selegiline+1 μM docetaxel, 1 mM selegiline+1 μM docetaxel).
For docetaxel and combination treatments, a 0.01% dimethyl sulfoxide (DMSO) solution prepared with complete RPMI medium (10% FBS, 100 U/ml Penicillin/Streptomycin; solution for culturing and treating PC3 cells) was used as a control. This was used as a solvent control, corresponding to the use of DMSO to prepare a 10 mM docetaxel stock solution. Thus, the 1 μM docetaxel treatments we used also contained 0.01% DMSO.
The change in cell viability induced by selegiline, docetaxel, and combinations thereof was measured after 48 hours with the CellTiter-Glo kit as previously described.
The experiments were performed in 3 different series, and for evaluation they were combined with the previously described normalization. Briefly, during the evaluation, we normalized to the mean of the untreated control in the given experiment, so the mean of the control was 100% and the percentage of each individual data point relative to the mean of the control was calculated accordingly. Because treatments with selegiline and docetaxel alone or in combination were performed in one experiment, we normalized to the mean of the untreated control without DMSO. Subsequent statistical analysis can be used to compare the results of simple and combined treatments and to determine whether DMSO as a solvent affects the viability of PC3 cells.
Statistical analysis was performed using GraphPad 8 software. A Shapiro-Wilk and Kolmogorov-Smirnov normality test was performed on the data set, followed by a one-way ANOVA test with a Holm-Sidak's multiple comparison test.

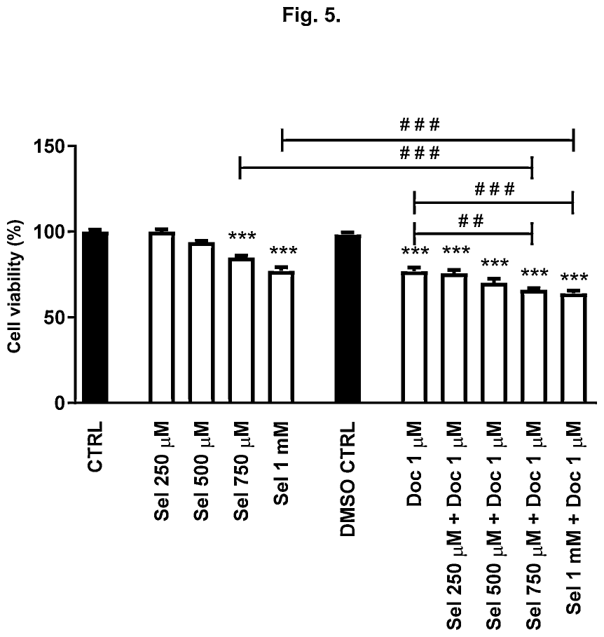

Results and Conclusion:
Selegiline reduces the viability of PC3 cells in a concentration-dependent manner over the concentration range of 250 μM to 1 mM. In the present series of experiments with selegiline, on the one hand, at a concentration of 1 mM, the viability-reducing effect already described in Example 1 was reproduced, on the other hand, it was also shown that a significant decrease in viability was observed even at a concentration of 750 μM. The statistical analysis confirmed that there was no significant difference in cell viability between untreated control and DMSO control. Based on the experimental data, the 1 μM concentration of docetaxel and all of the tested combinations of selegiline and docetaxel significantly reduced cell viability compared to the DMSO control. The results of the combination experiments showed that the combination of 750 μM selegiline+1 μM docetaxel and 1 mM selegiline+1 μM docetaxel had a significantly greater viability-reducing effect than 750 μM or 1 mM selegiline alone or 1 μM docetaxel alone (FIG. 5).
It is clear from this series of experiments that the selegiline and docetaxel in the studied combinations synergistically potentiate the PC3 viability reducing effect of each other.
The results are shown in FIG. 5. The results shown in the bar graph show that selegiline, docetaxel, and combinations thereof reduce PC3 cell viability in a concentration-dependent manner. CellTiter-Glo viability assay results after 48 hours of treatment. Statistical analysis: one-way ANOVA, Holm-Sidak's multiple comparison test, selegiline: Sel, docetaxel: Doc; CTRL: untreated control; DMSO CTRL: control using DMSO; ***p<0.001 vs. the given control group, ##p<0.01, ###p<0.001 vs. different treatment groups.
For the untreated CTRL, DMSO CTRL, selegiline 1 mM, docetaxel 1 μM, selegiline 1 mM+docetaxel 1 μM groups, N=10; selegiline 250 μM, selegiline 500 μM, selegiline 750 μM, selegiline 250 μM+docetaxel 1 μM, selegiline 500 μM+docetaxel 1 μM N=7; for selegiline 750 μM+docetaxel 1 μM N=6.

### Example 4

Treatment of Prostate Carcinoma in Dog

The results of the treatment using the invention are described below.

In dogs, drug treatment of prostate carcinoma takes place with a chemotherapeutic agent depending on the stage of the disease, e.g. with a platinum composition and optionally in combination with an anti-inflammatory of steroid or non-steroidal type, e.g. a cyclooxygenase enzyme inhibitor. With this therapy, there is usually a small, short-term (approx. 20 to 30 days) moderate and only temporary improvement in the typical symptoms of the disease (difficulty in urinating and increased prostate volume, deteriorating general condition). The effectiveness and limitations of the treatment are described in detail in two excellent publications. Clinical trial in dogs diagnosed with urogenital carcinoma (S D Allstadt, C O Rodriguez Jr., B. Boostrom, R B Rebhun, and K A Skorupski, Randomized Phase III Trial of Piroxicam in Combination with Mitoxantrone or Carboplatin for First-Line Treatment of Urogenital Tract Transitional Cell Carcinoma in Dogs, J Vet Intern Med 2015; 29: 261-267) suggests that in such cases, combination therapy with carboplatin or mitoxantrone and piroxicam (a non-steroidal anti-inflammatory drug) should be used as the primary therapy. However, the tumor, which also includes the prostate, significantly limits the effectiveness of treatment and both the progression-free phase and the median overall survival phase (109 days) are significantly shortened compared to urogenital carcinoma cases without prostate involvement. According to a recent review of the treatment of dogs with prostate carcinoma (S. Ravicini 1, S J Baines, A. Taylor, I. Amores-Fuster, S L Mason, E. Treggiari, Outcome and prognostic factors in medically treated canine prostatic carcinomas: A multi-institutional study, Vet Comp Oncol. 2018; 16: 450-458), using a combination of a chemotherapeutic agent and a non-steroidal anti-inflammatory drug, the progression-free phase is 76 days and the median survival phase is 106 days. However, even in the progression-free phase, unpleasant urogenital symptoms (e.g., difficulty urinating) and even treatment-related adverse drug reactions (e.g., gastrointestinal symptoms) typically occur, which can usually become more severe over time.

Veterinary Clinical Case Report: Treatment of Canine Prostate Carcinoma.

Treated Animal:

male dog (name: ‘Milo’), breed: fox terrier, age: 9.5 years

Clinical History:

Urinating problems lasting for a month: the dog has difficulty in urinating, emptying is the most difficult in case of full bladder, Defecation is also impeded, the stools are thin but shaped. There are no other complaints.

Clinical Examination:

Good general condition. Mucosa pale pink. Tactile lymph nodes are physiological in size. Touching the abdomen does not cause pain.

Imaging Examination:

Abdominal ultrasound: enlarged prostate

(current prostate volume: 25.60 cm³; physiological volume: 7.24 cm³)

Lumbar vertebra x-ray: One-way, LL image. The bone is intact, no lesion is depicted.

Calcified islands of the prostate are recognizable.

Cytological diagnosis (sampling through the abdominal wall): Prostate carcinoma ‘grade II-III’.

Drug Therapy:

Standard therapy (carboplatin, chemotherapeutic agent+firocoxib, COX-2 antagonist)+selegiline

Treatment Protocol:

Chemotherapy: carboplatin 300 mg/m² intravenously every 3-4 weeks for a total of six times.

Antiemetic: Emetron: 0.15 mg/kg intravenously 2×½ daily, if necessary for 3 days.

Anti-inflammatory/analgesic: firocoxib 227 mg tablets 1×¼ tablet daily.

Selegiline: 5 mg tablets, 2×1 tablet daily (10 mg/day).

The last two drugs were continued for 150 days.

Follow-up of clinical status on a weekly basis with a control clinical examination as described above.

Clinical Outcome

Continuous improvement of urinary symptoms and imaging diagnostic parameters (prostate volume approaching normal size) and consistently good general condition, asymptomatic on day 150 (progression-free).

The above example demonstrates that the use of selegiline in addition to a chemotherapeutic agent for the treatment of canine prostate carcinoma provides a particularly beneficial therapeutic effect over treatment without selegiline, resulting in even complete and permanent resolution of adverse clinical symptoms.

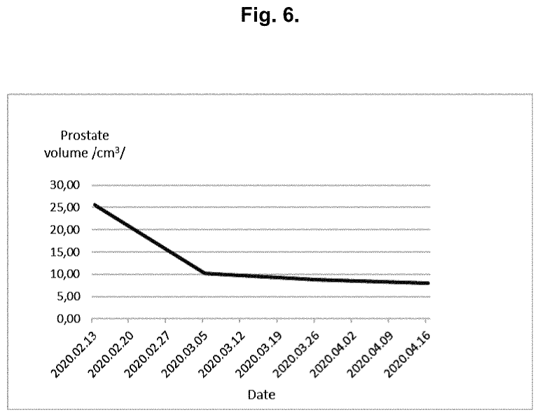

The results corresponding to the condition followed up to day 60 are shown in FIG. 6. The graph shows how prostate volume decreased in the treated animal during the first 60 days.

1. A pharmaceutical composition for prevention or treatment of prostate carcinoma (PCa) comprising a selective monoamine oxidase-B (MAO-B) inhibitor compound as sole active ingredient or in combination with other active ingredient, wherein the other active ingredient is selected from the group consisting of taxane acting on the microtubule system, preferably docetaxel or cabazitaxel (optionally in combination with a steroid); platinum preparations, preferably carboplatin (optionally in combination with a non-steroidal anti-inflammatory drug); topoisomerase inhibitors (optionally in combination with a non-steroidal anti-inflammatory drug); hormone therapeutic agents such as abiraterone or enzalutamide; androgen deprivation agents; androgen receptor agents; kinase inhibitors; antiangiogenesis agents; immunotherapeutic preparations; biological preparations having anticancer effects, anticancer preparations made from natural substances, e.g. anticancer preparations made from herbs; and compositions for inhibiting bone metastasis.

2. The pharmaceutical composition according to claim 1, wherein the MAO-B inhibitor compound is the sole active ingredient.

3. The pharmaceutical composition according to claim 2, wherein the MAO-B inhibitor compound is selected from the group consisting of selegiline and rasagiline, preferably selegiline.

4. The pharmaceutical composition according to claim 1, wherein the PCa is castration-resistant prostate carcinoma (CRPC).

5. The pharmaceutical composition of claim 1 comprising the selective MAO-B inhibitor compound in combination with the other active ingredient.

6. The pharmaceutical composition according to claim 5, wherein the other active ingredient for treating PCa is a taxane.

7. The pharmaceutical composition according to claim 5, wherein the other active ingredient for treating PCa is a hormone therapeutic agent.

8. The pharmaceutical composition according to claim 5, wherein the other active ingredient for treating PCa is a platinum preparation.

9. The pharmaceutical composition according to claim 5, wherein the MAO-B inhibitor compound is selected from the group consisting of selegiline and rasagiline.

10. The pharmaceutical composition according to claim 9, wherein the MAO-B inhibitor compound is selegiline.

11. The pharmaceutical composition according to claim 6, wherein the MAO-B inhibitor compound is selegiline and the taxane is docetaxel.

12. The pharmaceutical composition according to claim 5, wherein the PCa is castration-resistant prostate carcinoma (CRPC).

13. A method of preventing or treating prostate carcinoma (PCa) comprising administering to a human or animal in need thereof a selective MAO-B inhibitor compound alone as active ingredient in a pharmaceutically effective amount.

14. A method of preventing or treating prostate carcinoma (PCa) comprising administering to a human or animal in need thereof a selective MAO-B inhibitor compound in combination with other active ingredient for the treatment of PCa selected from the group consisting of taxane derivatives acting on the microtubule system, preferably docetaxel or cabazitaxel (optionally in combination with a steroid); platinum preparations, preferably carboplatin (optionally in combination with a non-steroidal anti-inflammatory drug); topoisomerase inhibitors (optionally in combination with a non-steroidal anti-inflammatory drug); antitumor compositions with a complex mechanism of action, such as mitoxantrone (optionally in combination with a non-steroidal anti-inflammatory drug); hormone therapeutic agents such as abiraterone or enzalutamide; androgen deprivation agents; androgen receptor agents; kinase inhibitors; antiangiogenesis agents; immunotherapeutic preparations; anti-inflammatory drugs; biological preparations having anticancer effects, anticancer preparations made from natural substances, e.g. anticancer preparations made from herbs; and compositions for inhibiting bone metastasis.

15. Method according to claim 13 or 14, wherein the MAO-B inhibitor compound is selected from the group consisting of selegiline and rasagiline.

16. Method according to claim 15, wherein the MAO-B inhibitor compound is selegiline.

17. Method according to any of claims 14 to 16 claim 14, wherein the MAO-B inhibitor compound is selegiline and the taxane derivative is docetaxel.

18. (canceled)
